(19) United States
(12) Patent Application Publication
(10) Pub. No.: US 2023/0248455 A1
(43) Pub. Date: Aug. 10, 2023

(54) SYSTEMS AND METHODS FOR AUTONOMOUS MEDICAL INTERVENTION

(71) Applicant: Duke University, Durham, NC (US)

(72) Inventors: Siobhan Oca, Durham, NC (US); Guangshen Ma, Durham, NC (US); Daniel Buckland, Durham, NC (US); Patrick Codd, Durham, NC (US)

(21) Appl. No.: 18/166,111

(22) Filed: Feb. 8, 2023

Related U.S. Application Data
(60) Provisional application No. 63/307,668, filed on Feb. 8, 2022.

(51) Int. Cl. A61B 34/32; A61B 34/10

(52) U.S. Cl. CPC A61B 34/32; A61B 34/10; A61B 2034/107; A61B 2034/2063

(57) ABSTRACT

A method for performing autonomous peripheral vascular localization, including: providing a robotic system including a camera and an ultrasound probe each connected to a robotic arm; moving the robotic arm such that the camera is positioned above and/or adjacent a target surface of a body part; capturing a three-dimensional (3D) image of the target surface using the camera; generating a scanning trajectory on the target surface using the 3D image; and scanning the ultrasound probe along the scanning trajectory by moving the robotic arm to autonomously localize a target vessel in the body part.

### RELATED APPLICATIONS

This application claims priority from U.S. Provisional Patent Application Serial No. 63/307,668, filed Feb. 8, 2022, the disclosure of which is incorporated by reference in its entirety.

### FEDERAL FUNDING LEGEND

This invention was made with Government support under Federal Grant no. 0NSSC20K1433 awarded by the National Aeronautics and Space Administration (NASA). The Federal Government has certain rights to this invention.

### BACKGROUND

A crucial step in the diagnosis or treatment of a patient is access to the blood in their veins. For vessels that are hard to identify at the skin surface, clinicians use an ultrasound (US) to identify and track veins for catheter placement. Although generally effective, this method requires a trained administrator. In some environments, such as remote scientific stations or understaffed hospitals, this resource may not be readily available. Thus, there is an ongoing opportunity for autonomous vessel localization and injection, which can provide an accurate and reliable process for patients who need to self-administer a treatment.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides all that is described and illustrated herein.

Some embodiments of the present disclosure are directed to a method for performing autonomous peripheral vascular localization, the method including: providing a robotic system comprising a camera and an ultrasound probe each connected to a robotic arm; moving the robotic arm such that the camera is positioned above and/or adjacent a target surface of a body part; capturing a three-dimensional (3D) image of the target surface using the camera; generating a scanning trajectory on the target surface using the 3D image; and scanning the ultrasound probe along the scanning trajectory by moving the robotic arm to autonomously localize a target vessel in the body part.

In some embodiments, the robotic system further includes a needle connected to the robotic arm and a catheter connected to the robotic arm. The method may further include: guiding the needle into the target vessel; and then deploying the catheter into the target vessel.

In some embodiments, the method further includes retracting the needle from the target vessel simultaneously with deploying the catheter.

In some embodiments, guiding the needle into the target vessel includes guiding the needle into the target vessel at a first angle relative to horizontal, the method further including rotating the needle while the needle remains in the target vessel such that the needle is at a second angle relative to horizontal that is smaller than the first angle, and wherein deploying the catheter is carried out at the second angle.

In some embodiments, scanning the ultrasound probe includes modulating a force of the ultrasound probe against the target surface to maintain substantially constant pressure against the target surface. Modulating the force of the ultrasound probe may be carried out using a proportional-integral-derivative (PID) controller.

In some embodiments, scanning the ultrasound probe to autonomously localize the target vessel includes detecting the target vessel and tracking the detected target vessel.

In some embodiments, tracking the detected vessel includes identifying a contour and center of the detected vessel.

In some embodiments, the detecting is carried out using machine learning and the tracking is carried out using active contour and Kalman filter.

In some embodiments, the moving, capturing, generating, and scanning steps, and optionally the guiding and deploying steps, are carried out automatically without human or manual input.

Some other embodiments of the present disclosure are directed to a system for performing autonomous peripheral vascular localization, the system including: a robot comprising a robotic arm; a camera connected to the robotic arm, the camera configured to capture a three-dimensional (3D) image of a target surface of a human body part; and an ultrasound probe connected to the robotic arm, the ultrasound probe configured to scan the target surface along a scanning trajectory established on the 3D image to localize a target vessel in the body part. The system is configured to autonomously: move the camera adjacent the target surface using the robotic arm, capture the 3D image, generate the scanning trajectory, and scan the target surface along the scanning trajectory using the robotic arm.

In some embodiments, the system further includes: a needle connected to the robotic arm; a catheter connected to the robotic arm; a needle linear actuator configured to advance the needle into and retract the needle out of the target vessel; and a catheter linear actuator configured to deploy the catheter into the target vessel, optionally concurrently with the needle being retracted out of the target vessel.

In some embodiments, the system further includes a rotational actuator configured to rotate the needle between a first angle relative to horizontal for inserting the needle into the target vessel and a second angle relative to horizontal for retracting the needle from the target vessel and deploying the catheter into the target vessel, wherein the first angle is 30-40 degrees and the second angle is 0-10 degrees.

In some embodiments, the system further includes a controller configured to modulate a force of the ultrasound probe against the target surface. The controller may include a PID controller.

The accompanying Figures and Appendix are provided by way of illustration and not by way of limitation. The foregoing aspects and other features of the disclosure are explained in the following description, taken in connection with the accompanying example figures (also “FIG.”) relating to one or more embodiments.

### BRIEF DESCRIPTION OF THE DRAWINGS

FIG. 1A is a perspective view of a robotic system for performing autonomous peripheral vascular location.

FIG. 1B is another perspective view of the system of FIG. 1A.

FIG. 2 is a flowchart illustrating the pipeline of the ultrasound and robotic guided vascular localization. The labelled Path-A, Path-B and Path-C are the individual scanning regions for each precision test. Thread 1 and 2 represent a parallel procedure of robot control and vision tracking. AC-Kalman denotes the Active-Contour-Kalman-Filter vision framework for vessel localization. The last image on the right side shows the surface point cloud and 3D reconstructed vascular contours.

FIGS. 3A-3F illustrate that the ultrasound-guided robotic system requires spatial calibration between the robot end-effector and the ultrasound image plane. We refer to the N-wire US calibration method and made improvements through design of a new calibration stage. The N-wire calibration uses a phantom with N cross-section wires that can be detected by a freehand US probe. However, this method is limited by a requirement of an external optical tracker as well as a complex design of a calibration stage. We introduce an improved US calibration method with a simpler design of the calibration stage (FIG. 3C).

FIG. 4 is a block diagram of the PID force controller. p (tᵢ₊₁) and \vec{ν}(tᵢ₊ᵢ) are the new position and orientation for the IK solver, and q is the corresponding robot configuration.

FIG. 5A illustrates 3D trajectory with surface points and normals at each time step.

FIG. 5B illustrates an example 2D model for the force control. Δd₂<Δd₁ depicts the adjustment based on the force input. \vec{V_{N1}} and \vec{V_{N2}} are surface normals from the original surface. \vec{F₁} and \vec{F₂} are force measurements.

FIG. 6 illustrates vessel detection, tracking and labelling in 2D US frame. The reference center and contour are labeled and compared with the predicted results. T is the time step and six sampled images are selected for visualization.

FIG. 7A illustrates visualization of RGB-D point cloud with surface trajectories created by manually selecting 3 regions (A₁, B₁ and C₁) and a zigzag trajectory generated automatically. Specifically, sections A₂, B₂ and C₂ are three different sections defined in the zigzag path.

FIG. 7B illustrates precision and demonstration variance results shown relative to vein radius.

FIGS. 8A-8C illustrate that the system can be used for non-invasive diagnostic scanning of breast tissue.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human who is undergoing a medical procedure using a system or method as prescribed herein.

The term “automatically” means that the operation can be substantially, and typically entirely, carried out without human or manual input, and is typically programmatically directed and/or carried out. The term “electronically” includes both wireless and wired connections between components. The term “programmatically” means that the operation or step can be directed and/or carried out by a digital signal processor and/or computer program code. Similarly, the term “electronically” means that the step or operation can be carried out in an automated manner using electronic components rather than manually or using merely mental steps.

Unless otherwise defined, all technical terms used herein have the same meaning as commonly understood by one of ordinary skill in the art to which this disclosure belongs.

An important step in medical care and research of astronauts involves testing blood or giving intravenous medications. Placing a catheter into a blood vessel takes some degree of training and takes up crewmember's time. One aspect of the present disclosure provides an automated technology that can identify blood vessels (such as a vein or artery), place a needle into the blood vessel, and then place a catheter into the blood vessel so that blood samples can be removed, and medications administered. The technology can reduce the amount of training needed to operate provide medical intervention. In an example embodiment, the system comprises a robotic arm with an attached ultrasound probe to automatically identify blood vessels using computer vision techniques, and then uses that same robotic arm to insert the needle and catheter.

More specifically, the system comprises an ultrasound-guided robotic system to track across tissue surfaces while maintaining acceptable contact forces. An end effector places a needle tip at the vessel centroid, and a motorized system rotates the needle to an angle that is appropriate for catheter deployment. The system can then be actuated to deploy a venous needle.

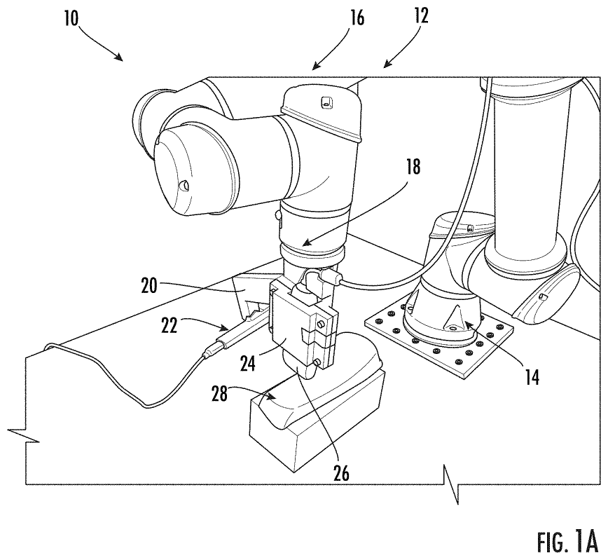

A robotic system 10 for performing autonomous peripheral vascular localization is illustrated in FIGS. 1A and 1B. The system 10 includes a robot 12 including a base 14 and a robotic arm 16. The robotic arm 16 may include an end effector 18. A camera holder 20 is connected to the robotic arm 16 and is configured to hold a camera 22. In some embodiments, the camera 22 is an RGB-D camera. An ultrasound probe holder 24 is connected to the robotic arm 16 and is configured to hold an ultrasound probe 26.

The system 10 is configured for autonomous peripheral vascular localization. As described herein, the robotic arm 16 is configured to precisely move the camera 22 and the ultrasound probe 26 to perform the localization. The robot 12 may include one or more controllers to direct the localization process automatically and programmatically.

FIG. 1A illustrates a phantom 28 of a human body part such as an arm. The robot 12 is configured to move the camera 22 above and/or adjacent the phantom 28 to capture a three-dimensional (3D) image of a target surface of the phantom 28. The robot 12 (or a controller associated therewith) is configured to generate a scanning trajectory on the target surface using the 3D image. The robot 12 is configured to move the ultrasound probe 26 to scan along the scanning trajectory to autonomously localize a target vessel in the body part.

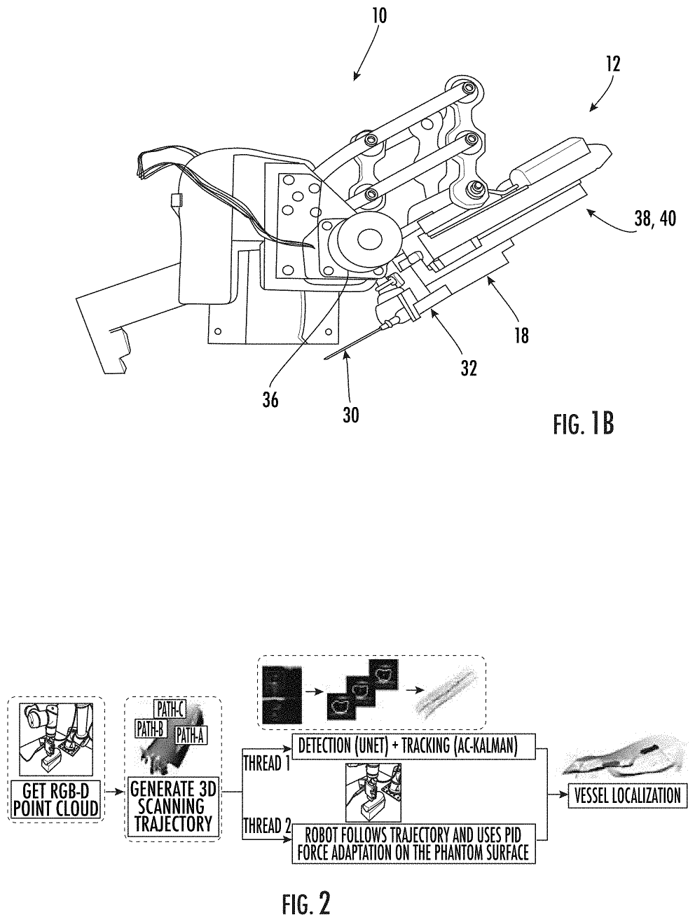

This procedure is illustrated in FIG. 2. First, the system takes a depth image of the target surface used to generate the trajectory position. Second, the trajectory is either explicitly chosen or automatically placed on the surface. Next, in one thread, the robot follows the predetermined trajectory and uses force adaptation (e.g., PID force adaptation) to keep contact of the ultrasound probe with the surface, which is critical for visualization. Meanwhile, the images are analyzed in real time for initial detection using, for example, U-net and then tracked using, for example, an active contour-Kalman filter to collect the centroids of the vessel. Finally, this data is transformed to generate the vessel locations in the robot base frame coordinates.

As described further herein, a controller such as a PID controller (FIG. 4) can be used to enable the robot to move smoothly with a safe force threshold on the arm phantom.

Referring to FIG. 1B, the system 10 may provide a robotic mechanism to autonomously guide a needle tip into a target vessel and deploy a peripheral catheter with ultrasound guidance. A needle 30 and a catheter 32 are held on the end effector 18 of the robot 12. The needle 30 is rotated from a relatively large angle (e.g., 30-40° relative to horizontal) that is ideal for vein puncture to a smaller angle (e.g., 0-10° relative to horizontal) that is better to thread the catheter 32 into the vein. For example, the needle 30 may extend for insertion around 30° and then a rotational motor or actuator 36 may rotate the needle to around 10° while the needle tip remains in the vein.

After the change in angle, a linear catheter motor or actuator 38 may extend the catheter 32 as a linear needle motor or actuator 40 retracts the needle. The extension of the catheter 32 and the retraction of the needle 30 may occur simultaneously. Finally, the catheter 32 may be left in the arm for final securement by the human user or controller.

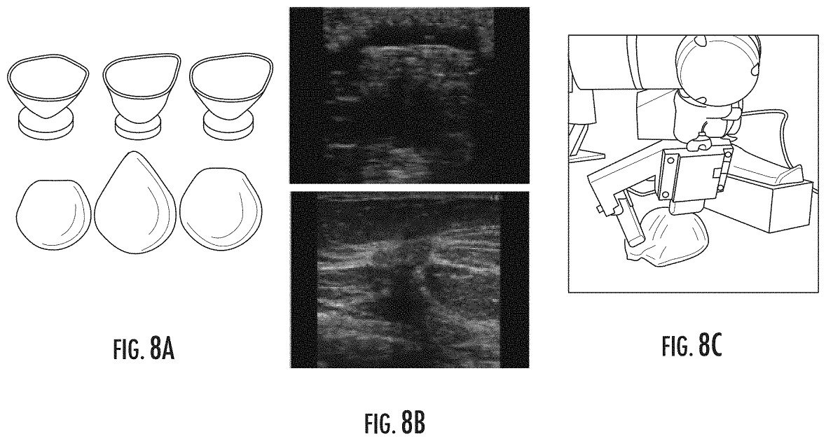

In another embodiment of the present disclosure, the robotic arm system can be used as a platform to provide other types of ultrasound-based diagnoses and therapeutics. In a non-limiting example, the system can be used for non-invasive diagnostic scanning of breast tissue. The system can be adjusted to provide appropriate surface pressure and motion planning. An example embodiment is in FIGS. 8A-8C.

Another embodiment of the present disclosure provides a method of providing medical intervention using a device as disclosed herein.

Additional details of the disclosed system and method are described in the following example.

### EXAMPLE

Described below is an autonomous RGB-D and 2D ultrasound-guided robotic system for collecting 3D localized volumes of peripheral vessels. This compact design, with available commercial components, lends itself to platform utility throughout the human body. The fully integrated system works with force limits for future safety in human use. A PID force controller is used for smooth and safe robot scanning following a priori 3D trajectory generated from a surface point cloud. System calibration is implemented to determine transformations among sensors, end-effector and robot base. A vascular localization pipeline that consists of detection and tracking is proposed to find the 3D vessel positions in real-time. Precision tests are performed with both predesignated and autonomously selected areas in an arm phantom. The average variance of the autonomously collected ultrasound images (to construct 3D volumes) between repeated tests is shown to be around 0.3 mm, similar to the theoretical spatial resolution a clinical ultrasound system. This fully integrated system demonstrates the capability of autonomous collection of peripheral vessels with built-in safety measures for future human testing.

### Introduction:

A crucial step in the diagnosis or treatment of any patient is access to the blood in their veins. For vessels that are hard to identify at the skin surface, clinicians use an ultrasound (US) to identify and track veins for catheter placement. Autonomous robotic vessel localization, including detection and tracking via US guidance, can provide a more accurate and reliable process for patients, which does not require advanced training for the clinician.

Robotic controlled US has been widely employed in research, but differs greatly in intended use, autonomy from the human controller, and flexibility of use case. US guidance was used for multiple robot assisted medical procedures such as prostatectomy, breast biopsies and carotid artery tracking. Most of these platforms differ from the proposed system because they rely significantly on the controller and the clinician for expertise and input. Many systems either require a controller to identify the key targets of the system or utilize teleoperation with shared control placement of the probe. Therefore, the autonomy of these robotic systems is limited, as the presence of a clinician is necessary to navigate and identify correct structures. For intervention without a clinician, the system must be able to perform certain tasks autonomously. This is only possible if the robot-controlled US can safely navigate the surface of the body without injury-causing force on the patient's body, while accomplishing the objectives of the procedure.

Robotic controlled US systems that are autonomously force modulated have been developed, but many of these systems are specialized for a specific and limited surface of the patient. With the range and accuracy of current commercially available robotic arms, a more flexible system may be possible for different vision-based control tasks such as finding and tracking various vessels in the body through precise control of the US probe. Force modulation can be achieved with specific sensors on the end effector (EE) or as part of the system, as with the Universal Robotics UR5e. For example, Mathaissen et al. demonstrated that it is possible to use the UR5e robotic arm for US guided tele-robotic procedures.

Building on an effective robot controlled US, autonomous vascular localization mainly includes two tasks: vessel detection and vessel tracking. Vessel detection aims to search for a vessel, determine its location, and use this proposed region to initialize the vessel tracking process. Given a vessel candidate, the goal of vessel tracking is to find the 3D coordinates of the vessel contour in each frame and transform to the global coordinates, based on the unique features of the vessels in the US image. The main visual characteristics of the vasculature is lower pixel intensity inside the vessel boundary and higher intensity outside the edge, as the echoes reflected back to the US encounter different material properties between the blood, muscle, and fatty tissue. These unique features enable the use of Active Contour and its variants to track the object contours with a level-set formulation. Moreover, recent progress in deep learning shows good performance for various tasks of 2D image analysis, such as U-net, a powerful network for medical image segmentation. Integration of deep learning and Active Contour method shows potential applications for autonomous vascular scanning. Recent work proposed a Convolutional Long Short Term Memory network to segment the vessel by using temporal features, demonstrating the efficacy of deep learning for vessel analysis in US images.

Related work that found target peripheral vasculature includes a robotic venipuncture prototype which integrates a near-infrared and US imaging with a 7-degree-of-freedom needle insertion subsystem by Chen et al. However, this system is limited to surface vessels that can be identified by an infrared camera and requires a custom robotic system only used in cannulation of peripheral vessels. Chen et al. also proposed a deep learning strategy for fully autonomous vascular access with B-mode and color doppler image sequences by using a recurrent convolutional encoder-decoder network. This is different from our work since only the B-mode image sequences are used in the study. To address these limitations, the proposed system is comprised of an integrated US probe and RGB-D camera at the robotic arm EE. This system is able to capture an a priori point cloud, generate a scanning trajectory and perform a fully automatic vessel localization in real-time by adjusting the force with a PID controller. The main contributions of this example may be summarized as:

1. System integration of an ultrasound probe and an RGB-D camera to a 6-DOF robotic arm for performing autonomous peripheral vascular localization.
2. A PID control strategy that enables the robot to move smoothly with a safe force threshold on an arm phantom.
3. A 3D vessel localization system for automated vessel detection, tracking and contour reconstruction.

### Methods:

System Hardware

This system includes three main components: a UR5e robotic arm (Universal Robots, Denmark), Interson US probe (Interson Corporation, CA), and a Realsense SR305 RGB-D camera (Intel Corporation, CA), as shown in FIG. 1A. The US probe and RGB-D camera are attached to the robotic arm's EE with a 3D-printed stage, which has fixed the physical positions of the sensors. The built-in UR5e Force-torque is utilized to measure the robot force contacts.

The surface image is retrieved by the Intel Realsense SR305. With a range of 0.2-1.5 m and up to 640×480 resolution at 60 frames per second (fps), it sufficiently depicts the stationary surface of the tissue. This surface determines the orientation of the US probe for smooth and safe movement across the tissue for a given trajectory. An initial image is taken to capture the full surface, assuming no movement of the tissue throughout the procedure. This is required as the camera, connected to the robot arm EE, cannot have good visualization and be 0.2 m from the tissue surface during US collection. The US image data is collected with Interson's (SP-101) USB US Imaging Probe, with 7.5 MHz and 5 cm depth range. The low frequency (7.5 MHz) enables penetration to deeper tissue and it is widely used for vessel detection in human arm. Low frequency (LF) B-scan US video is converted to a sequence of images at 30 fps. Each LF image has dimension as 754×494 pixels. The resolution and speed of the US probe enable fast image analysis during the robot scanning procedure.

System Calibration

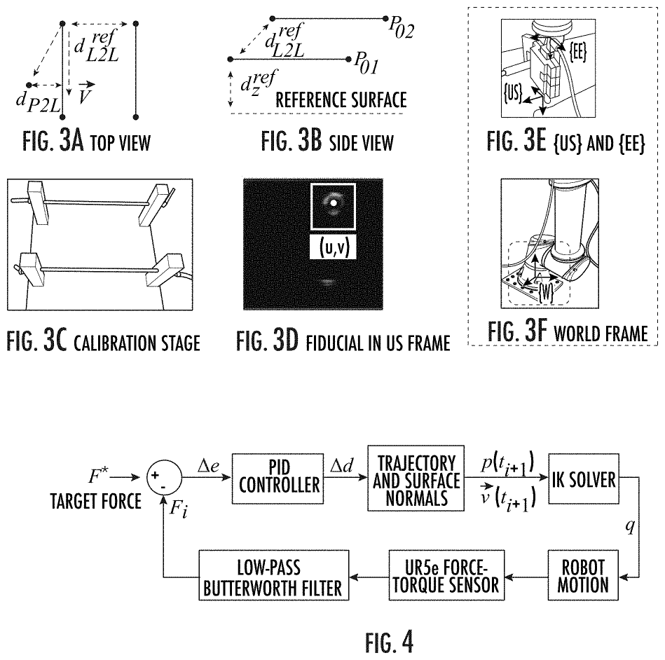

For successful vascular localization, system calibration aims to find precise transformations between various coordinate frames. The base of robotic arm is defined as the global world frame denoted as {W} (FIG. 3F). The frames of robot end-effector and ultrasound probe center tip are described as {EE} and {US} respectively (FIG. 3E). The goal of calibration is to find the frame transformations of T_{US}^{EE}=(R_{US}^{EE}, t_{US}^{EE}) and T_{EE}^{W}=(R_{EE}^{W},t_{EE}^{W}), such that a pixel such that a pixel p(u,v) in the ultrasound image plane can be transformed to {W}.

p^{W}=R_{EE}^{W}(R_{US}^{EE}[u*γ, ν*γ, 0]^{T}+t_{US}^{EE})+t_{EE}^{W}

where R_{EE}^{W} and R_{US}^{EE} are 3×3 rotation matrix, and t_{US}^{EE} and t_{EE}^{W} are 3×1 translation vectors. p^{W} is the 3D coordinate of a pixel defined in {US}. The coefficient γ is a ratio between the pixel distance and millimeter for US images. T_{EE}^{W} can be calculated by forward kinematics of the robot arm.

RGB-D Camera Calibration: RGB-D camera calibration is a common procedure in robotics with the aim of finding the optimal transformation between the depth camera and the robotic EE. The RGB-D camera calibration was implemented with an iterative approach to solve the Hand-Eye calibration problem.

Ultrasound Calibration: Ultrasound-guided robotic system requires spatial calibration between the robot end-effector and the ultrasound image plane. We refer to the N-wire US calibration method and made improvements through design of a new calibration stage. The N-wire calibration uses a phantom with N cross-section wires that can be detected by a freehand US probe. However, this method is limited by a requirement of an external optical tracker as well as a complex design of a calibration stage. We introduce an improved US calibration method with a simpler design of the calibration stage (FIG. 3C).

US calibration aims to find {EE} to {US} transformation such that the configuration of the reconstructed tube locations in the US image (FIG. 3D) matches the geometry of the calibration stage. To solve the US calibration problem, we formulate an optimization problem by defining three geometric error constraints: point-to-line E_{P2L} point-to-plane E_{P2Z} and line-to-line E_{L2L}. E_{P2L} penalizes the distance between the reprojection point and the tube center line; since the two tubes in the calibration stage are parallel to the table surface (reference plane), E_{P2Z} measures the length between the detected fiducial (at the tube) and the reference plane; E_{L2L} is the geometric constraint of two parallel tubes where the line-to-line distances should be fixed. US calibration requires a collection of sampled data from various robot configurations in 3D for the generality of the method. The optimization aims to minimize the summation of E_{P2Z}, E_{P2L} and E_{L2L} among all the sampled data:

$$arg {min}_{{T}_{US}^{E E} , \vec{V} , {P}_{o , 1} , {P}_{0 , 2}} {\sum }_{i = 1 , 2} {\sum }_{j = 1}^{m} {‖ {d}_{z} ( {P}_{i , j}^{W} ) - {d}_{z}^{ref} ‖}_{2} + {\sum }_{i = 1 , 2} {\sum }_{j = 1}^{m} {‖ ( {P}_{i , j}^{W} - {P}_{o , i} ) - [ ( {P}_{i , j}^{W} - {P}_{o , i} ) \cdot \vec{V} ] * \vec{V} ‖}_{2} + \vert ( {‖ ( {P}_{o , 2} - {P}_{o , 1} ) - [ ( {P}_{o , 2} - {P}_{o , 1} ) \cdot \vec{V} ] * \vec{V} ‖}_{2} - {d}_{L 2 L}^{ref} ) \vert$$

Where P_{o,i} is the initial position of the i-th tube. P_{i,j}^{w} is computed by Eqn. 1 based on the fiducial pixel center (u, v) at the i-th tube and the j-th point (FIGS. 3B and 3D). \vec{V} represents the same direction vector of the two parallel tubes. d_{z}(P_{i,j}^{w}) is the Z-axis coordinate to denote the point-to-plane distance from the reference surface. d_{z}^{ref} describes the ground truth distance between the reference surface and the height of the tube. Similar to d_{z}^{ref}, d_{L2L}^{ref} depicts the fixed reference distance between the two parallel tubes. The formulation in (2) includes 15 unknown parameters and the derivative of the objective function can be approximated by Finite-difference. The Python Scipy Optimization Package is used to find the optimal solution via Broyden-Fletcher-Goldfarb-Shanno (BFGS) method. With a reasonable initial guess, a local minimum can be determined.

Robot Control Strategy

Autonomous vascular localization requires a robust robot control strategy to move the US probe on a deformable surface. The robot must minimize jolting movement and continuously adapt the force to maintain constant pressure on the surface. Physical robot movement follows a surface trajectory, with proposed positions and normals generated from the a priori point cloud data, and force control, which adapts dynamically to the surface-based forces in the EE force torque sensor.

The adjustment of the trajectory via force input is critical for safe scanning to avoid potential deformation of the vessel in the arm. A robust PID force controller can maintain adequate contact to the surface based on the point cloud data with noise and bias in visual perception. Therefore, we propose a vessel scanning approach using a PID controller and an a priori surface point cloud.

Trajectory from Point Cloud: The system first moves to an initial position and takes an RGB-D image of the arm phantom. The depth data is filtered to eliminate outliers and segmented to select points that lie above the table surface by at least 5 cm. The processed point cloud is denoted as \vec{P}={p₁, p₂, . . . , pₙ}, pₙ ∈ R³. The surface normals are estimated from \vec{P} and referred as \vec{V_{N}}={\vec{ν₁}, \vec{ν₂}, . . . , \vec{νₙ}}, \vec{νₙ} ∈ R³. A pre-defined 3D trajectory is applied to the surface point cloud data. To eliminate jerky motion and generate a smooth trajectory, a basis spline (B-spline) function is made from the surface positions P and normals \vec{V_{N}}. The interpolated curves are denoted as f_{P}(t): R→R³ and f_{N}(t): R→R³ with t ∈ [0, 1] as the ratio of the time step.

Force PID control: The force is modulated by adjusting the position of the US probe towards the surface normal f_{N}(t) at the 3D trajectory f_{P}(t), and we refer this offset distance as penetration depth which is denoted as Δd. The target force is set as F_{target}=3.5 N with a danger force threshold defined as 5.0 N. A PID controller takes the force error as input and outputs the penetration depth for the next move (FIG. 4). To determine the current force with sensory noise, a 5th order low-pass Butterworth filter is applied to continuously utilize the past 50 measurements for force control.

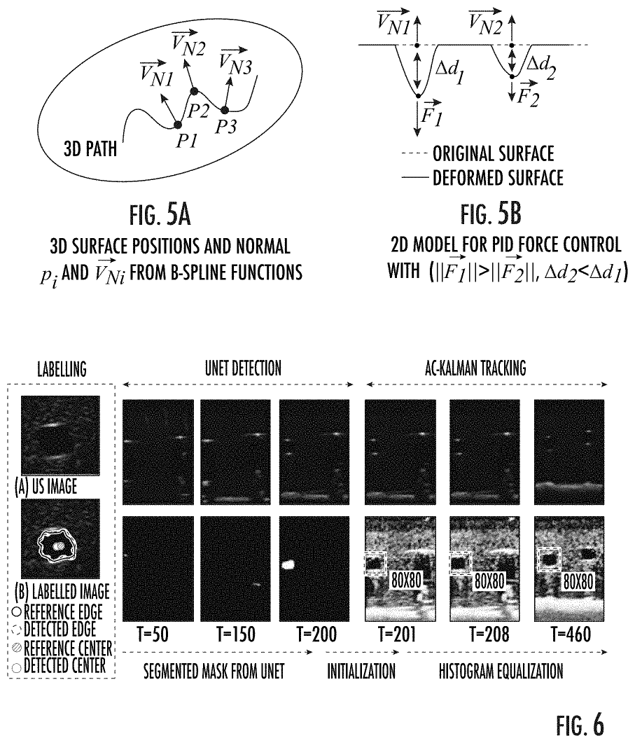

For the consecutive motions in the same trajectory, we have p(tᵢ₊₁)=fₚ(tᵢ₊₁) +Δd·\vec{ν}(tᵢ₊ᵢ) with \vec{ν}(tᵢ₊₁)=f_{N}(tᵢ₊ᵢ). Δd is the control output from the PID controller based on force error. The controller adjusts the error between the current and the target force by modulating A d at each time step. For example, FIG. 5B illustrates a simple 2D projection model for the PID controller where the force can be adjusted by controlling the penetration depth.

Furthermore, the position of the inverse kinematic target satisfies the constraint that the US probe aligns with the surface normal and the orientation follows the guided direction, i.e. parallel to the world frame's Y-Z plane. With the updated trajectory, the inverse kinematic solver of Klampt Software Toolbox is used to generate the joint configurations for robot movements.

Vessel Localization System

An effective robot control strategy can usually ensure a successful vascular localization by keeping adequate contacted areas and smooth movements. The US frames are consistently collected during robot scanning and the vessel localization pipeline mainly consists of two problems: vessel detection and tracking (FIG. 6). These problems can be modelled as image segmentation tasks with the vessel contours as targets. U-net network architecture has shown great success for image segmentation tasks and is implemented to detect the first vessel candidate from the raw US frames. The output of U-net is a masked image with clustered pixel regions and the centroid of the segment can be computed by averaging the connected pixel coordinates.

Given a vessel candidate for tracking initialization, the image is first pre-processed by histogram equalization to increase the contrast of the grayscale intensity inside and outside the vascular structure. An 80×80 region of interest (ROI) is centered around the detected vessel centroid, which is empirically determined in order to cover the region of the vascular contour. After tracking initializes, the next task is to segment the vessel boundary in the ROI via the Active Contour method. Chan-Vese model for active contours is a powerful tool to localize the boundary for the US images that cannot be easily processed by simple threshold-based or gradient-based methods. In the ROI, the Chan-Vese active contour model is able to flexibly identify the vessel boundary for vessel tracking. With the segment pixels, the center of the vessel can be tracked in real-time and a temporal Kalman filter is employed to trace the centers, which performs an accurate state estimation under inaccurate vessel tracking measurements. The Active-Contour-Kalman-Filter framework is referred as “AC-Kalman” in this study. Since the image processing is only analyzed in a local ROI and the change of boundary between the consecutive frames is small, the number of iterations for the active contour model is set as 5 for real-time application.

### Experiments and Results

Testing of Sensor Calibration

Camera Calibration: The evaluation metric of the camera calibration was based on the reprojection errors of optimal transformation. 30 different configurations were defined to collect the images with the RGB-D camera on the robot controlled EE. Average reprojection error was 2.5 mm.

Ultrasound Calibration: The optimal transformation between {US} and {EE} was obtained by US calibration. The ultrasound calibration was evaluated by applying the optimal TH to new data points and calculating the reprojection errors based on E_{P2L}, E_{P2Z} and E_{L2L}. The optimal value of E_{P2L} should be zero since all the center points are localized at the center line of the tube. E_{P2Z} and E_{L2L} are compared with the reference values for validation.

To evaluate the US calibration, 12 datasets were generated by moving the calibration stage to different 3D positions and used for calculating the reprojection errors in E_{P2L}, E_{P2Z} and E_{L2L}. These data were collected from each tube by moving the phantom to 6 different positions (different X, Y, Z coordinates), including 10 data points for each tube. The Root-mean-square errors of E_{P2L}, E_{P2Z} and E_{L2L}are reported as 0.61 mm, 0.33 mm and 0.76 mm, with the maximum errors as 1.24 mm, 0.76 mm and 1.20 mm. This shows that the calibration method can find the precise transformation between sensor frames with good accuracy.

### Vessel Detection and Tracking Experiments

Detection and tracking have the same tasks of tracing the closed vessel boundary and estimating the centers. Based on the testing dataset, the results of the vision pipeline are compared with the labelled reference contours and centers. DICE similarity coefficient and the offset of the center error were used to evaluate the vision performance (FIG. 6). The center error measures the distance between the detected and reference centers, while the DICE evaluates the similarity between two regions formed by the traced vessel boundary and the labelled contour.

Testing data used for vessel detection and tracking were different. For the detection dataset, the robotic system was controlled to scan the arm phantom multiple times to search many areas of the phantom. A small dataset was sufficient for the phantom study since the image features were similar in different frames. Then, 100 images were randomly sampled from the collected data and shuffled, which include zero or more vessels. For the tracking dataset, three specific regions in the phantom, which show only one vessel, were chosen manually and the consecutive US image sequences were utilized for the tracking experiments. For each region, more than 30 images in different time steps were selected from the consecutive image sequences. This ensured all the sampled images were from the same tracking process. A total of 100 images were used for evaluation of the tracking method.

Table I shows the results of average DICEs and center errors. The success rate is defined by counting the ratio of the correct detection or tracking case with a center error less than 1.0 mm. The image processing time for vessel detection is 12 fps with GPU processing and 32 fps for the vessel tracking with CPU. The tracking speed with 32 fps demonstrates that the vision prototype can perform real-time vessel localization (32 fps >24 fps) in a phantom.

TABLE I
Vision testing results with US phantom images

| | Detection | Tracking |
|---|---|---|
| Average center error (mm) | 0.46 | 0.51 |
| Average DICE coefficient | 0.88 | 0.85 |
| Success Rate | 96% (96/100) | 98% (98/100) |
| Image Processing Time (fps) | 12 | 32 |

Precision Experiment

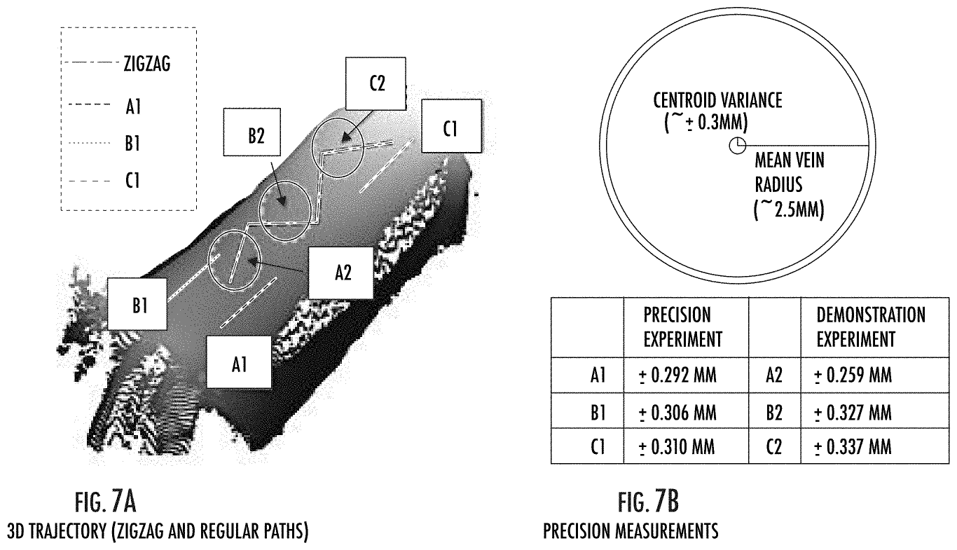

The goal of the precision experiment was to test the system precision on an arm phantom with the proposed vascular localization system. First, a surface map was captured by an RGB-D camera and utilized to manually determine 3 locations for US scanning, referred A₁, B₁ and C₁, as shown in FIG. 7A. The arm phantom includes vessels with multiple structures, e.g. some vessels are fused in a single vessel and others have limited length. Therefore, sections were chosen with only one vessel for the precision experiment. A surface trajectory was generated based on these selections for repeated testing (4 times for each 3 locations shown in FIG. 7A).

For each scanning location, the robot moved along the predefined path and automatically adapted the force on the surface to minimize deformation and maintain adequate contacted area. The vessel centers and contours were tracked simultaneously during the real-time robot movement. This procedure was repeated for 4 trials for each scanning location with 12 cases conducted. The system performance was evaluated by the average variances among all 4 trials centroid locations collected. FIG. 7B illustrates the system precision results on each trajectory. The mean radius of the vessel in the phantom is around 2.5 mm and the variances are +0.3 mm.

Demonstration Experiment

Demonstration experiment aimed to validate the utility of the integrated system to develop a map of the phantom's vessels and perform a fully automatic vessel localization. Another goal was to show that the proposed PID controller can modulate the force safely on a curved surface. To scan the whole phantom surface, a zigzag trajectory was generated from the surface map with minimal a priori user settings, by defining the confined region of the set point at the path. Similar to the precision experiment, this demonstration was conducted with repeated measurements to ensure that the resulting map was repeatable and effective at defining the vessels on the automated trajectory.

To find the vessel centers for evaluation, US images were collected simultaneously during zigzag scanning. As the trajectory covered various surface regions and it could not guarantee that only one vessel always appears in the US images, three trajectory sections were manually selected so that at least one vessel appears consistently in the US images, referred as A₂, B₂ and C₂ (FIG. 7A). The four repeated trials were conducted with the same experimental setting and it was assumed that the small difference between each trial did not change the index of the image in each dataset. Therefore, the sample dataset should be the representation of the same trajectory section for the zigzag path. The centroids of these vessels were compared between demonstrations to ensure repeatability. The results of variance analysis are shown in FIG. 7B.

### Discussion and Conclusion

The precision experiment and demonstration validate the functionality of the proposed system including the PID controller as well as the vascular localization pipeline. The results of the precision experiment show a variability per location of approximately ±0.3 mm. As the pose repeatability of the UR5e robot is 0.03 mm, it is expected that the error in measurements comes primarily from US system and automated vision selection. Error due to the ultrasound probe itself is expected to be approximately 0.25 mm. Target blood vessels for procedures, such as peripheral vessel cannulation, would be around 4 mm. The error of the centroid of ±0.3 mm is acceptable within several margins of error for safety. If the cannulation or other device were part of the robot EE, this error would only be in addition to a needle insertion device. Precision error combined with calibration results would be maximum +1.8 mm of the complete system. Therefore, these results indicate the system has sufficient precision for 3D reconstruction of critical peripheral vasculature and could be used in conjunction with another intervention that requires this level of accuracy.

The demonstration aimed to mimic in vivo requirements with a safety threshold of 5N and target force as 3.5N, phantom curvature like that of a forearm and vessels of similar size (˜5 mm). Additionally, the system works autonomously as there is no human intervention from RGBD picture capture until vessel reconstruction. In total, the system demonstrates the capture of the phantom surface to perform a zigzag pattern of searching and reconstruction, at safe force, of multiple vessels in the phantom. This is done in real-time using a commercial robotic arm, linear probe and RGB-D camera. As shown in FIG. 7B, the precision of around 0.31 mm of the autonomously found vessels is similar to the manually directed precision experiment.

For the vision testing, the success rates for detection and tracking are 96% ( 96/100) and 98% ( 98/100). This demonstrates the proposed vascular pipeline can precisely localize the center and the contour of the phantom vascular structure. In addition, the average center error of 0.46 mm corresponds to a 7.0 pixel distance in the US image. This provides adequate error threshold for the detection task with a 80×80 image ROI. This indicates that if the detected center is located in the ROI, the AC-Kalman method can robustly track the position of the center in real-time. The average center error for tracking (0.51 mm) shows that the deviation contributes to 10% of the vessel dimension (the diameter of the vessel is about 5.0 mm), which is an acceptable range for successful vessel tracking.

In summary, this system demonstrates precise reconstruction of small tubes and their centroids deep below the curved surface, like the arm, within safe force limits. The vascular scanning procedure is performed with safety precautions that will be applicable to future human use.

Another aspect of the present disclosure provides all that is described and illustrated herein.

The systems and methods described herein can be implemented in hardware, software, firmware, or combinations of hardware, software and/or firmware. In some examples, the systems and methods described in this specification may be implemented using a non-transitory computer readable medium storing computer executable instructions that when executed by one or more processors of a computer cause the computer to perform operations. Computer readable media suitable for implementing the systems and methods described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system or method described in this specification may be located on a single device or computing platform or may be distributed across multiple devices or computing platforms.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

We claim:

1. A method for performing autonomous peripheral vascular localization, the method comprising:
providing a robotic system comprising a camera and an ultrasound probe each connected to a robotic arm;
moving the robotic arm such that the camera is positioned above and/or adjacent a target surface of a body part;
capturing a three-dimensional (3D) image of the target surface using the camera;
generating a scanning trajectory on the target surface using the 3D image; and
scanning the ultrasound probe along the scanning trajectory by moving the robotic arm to autonomously localize a target vessel in the body part.

2. The method of claim 1 wherein the robotic system further comprises a needle connected to the robotic arm and a catheter connected to the robotic arm, the method further comprising:
guiding the needle into the target vessel; and then
deploying the catheter into the target vessel.

3. The method of claim 2 further comprising retracting the needle from the target vessel simultaneously with deploying the catheter.

4. The method of claim 2 wherein guiding the needle into the target vessel comprises guiding the needle into the target vessel at a first angle relative to horizontal, the method further comprising rotating the needle while the needle remains in the target vessel such that the needle is at a second angle relative to horizontal that is smaller than the first angle, and wherein deploying the catheter is carried out at the second angle.

5. The method of claim 1 wherein scanning the ultrasound probe comprises modulating a force of the ultrasound probe against the target surface to maintain substantially constant pressure against the target surface.

6. The method of claim 5 wherein modulating the force of the ultrasound probe is carried out using a proportional-integral-derivative (PID) controller.

7. The method of claim 1 wherein scanning the ultrasound probe to autonomously localize the target vessel comprises detecting the target vessel and tracking the detected target vessel.

8. The method of claim 7 wherein tracking the detected vessel comprises identifying a contour and center of the detected vessel.

9. The method of claim 7 wherein the detecting is carried out using machine learning and the tracking is carried out using active contour and Kalman filter.

10. The method of claim 2 wherein the moving, capturing, generating, and scanning steps, and optionally the guiding and deploying steps, are carried out automatically without human or manual input.

11. A system for performing autonomous peripheral vascular localization, the system comprising:
a robot comprising a robotic arm;
a camera connected to the robotic arm, the camera configured to capture a three-dimensional (3D) image of a target surface of a human body part; and
an ultrasound probe connected to the robotic arm, the ultrasound probe configured to scan the target surface along a scanning trajectory established on the 3D image to localize a target vessel in the body part,
wherein the system is configured to autonomously: move the camera adjacent the target surface using the robotic arm, capture the 3D image, generate the scanning trajectory, and scan the target surface along the scanning trajectory using the robotic arm.

12. The system of claim 11 further comprising:
a needle connected to the robotic arm;
a catheter connected to the robotic arm;
a needle linear actuator configured to advance the needle into and retract the needle out of the target vessel; and
a catheter linear actuator configured to deploy the catheter into the target vessel, optionally concurrently with the needle being retracted out of the target vessel.

13. The system of claim 12 further comprising a rotational actuator configured to rotate the needle between a first angle relative to horizontal for inserting the needle into the target vessel and a second angle relative to horizontal for retracting the needle from the target vessel and deploying the catheter into the target vessel, wherein the first angle is 30-40 degrees and the second angle is 0-10 degrees.

14. The system of claim 11 further comprising a controller configured to modulate a force of the ultrasound probe against the target surface.

15. The system of claim 14 wherein the controller comprises a PID controller.
